(19) United States
(12) Patent Application Publication
(10) Pub. No.: US 2026/0115042 A1
(43) Pub. Date: Apr. 30, 2026

(54) Phacoemulsification Apparatus Including An Anti-Vacuum Surge Valve Mechanism

(71) Applicant: JOHNSON & JOHNSON SURGICAL VISION, INC., Irvine, CA (US)

(72) Inventors: Vadim Gliner, Haifa (IL); Assaf Govari, Haifa (IL)

(21) Appl. No.: 18/931,726

(22) Filed: Oct. 30, 2024

(51) Int. Cl. A61F 9/007

(52) U.S. Cl. CPC A61F 9/00745

(57) ABSTRACT

A phacoemulsification system including a phacoemulsification probe including a hollow needle configured to provide an aspiration channel, a valve module including a valve closing element configured to close the aspiration channel upon activation, and a controller for providing an electric current pulse to activate the valve closing element to close the valve module, wherein the controller is programmed to detect a vacuum surge, provide an electric current pulse to a valve actuator in order to activate the valve closing element to close the valve module, wherein the shape of the current pulse includes a first section configured to accelerate the valve closing element, and a second section to decelerate the valve closing element as the valve closing element approaches an end of travel to prevent the valve closing element from hitting the end of travel and bouncing back, thereby preventing a recoil effect. Related apparatus and methods are also described.

### TECHNOLOGICAL FIELD

The present disclosure relates to a phacoemulsification apparatus including a valve module such as an Anti-Vacuum Surge (AVS) module (also known as Chamber Stabilization System (CSS)), and, more particularly, but not exclusively, to an AVS valve mechanism enabling closing the valve as fast as possible while optionally preventing a recoil effect.

### BACKGROUND

Background art includes:

U.S. Pat. No. 11,771,818 to Govari et al., describes a fluid dynamics system which includes a solenoid valve having a valve body with ports, including an inlet port and an outlet port, and a valve cavity having a direction of elongation and is configured to provide fluid connectivity between respective ones of the ports; a solenoid coil disposed in the valve body around the valve cavity; a plunger including a permanent magnet that is configured to move back-and-forth along the direction of elongation between a first position and a second position in the valve cavity selectively controlling the fluid connectivity between respective ones of the ports; and a controller configured to apply at least one current to the solenoid coil to selectively move the plunger between the first position and the second position, and to selectively maintain the plunger in the first position and the second position.

The disclosures of all references mentioned above and throughout the present specification, as well as the disclosures of all references mentioned in those references, are hereby incorporated herein by reference.

### OVERVIEW

During phacoemulsification of an eye lens, emulsified lens particles are aspirated by an aspiration channel of the phacoemulsification apparatus. When a particle blocks an inlet of the aspiration channel (e.g., the distal end/port of a needle of a phacoemulsification probe) causing occlusion of the channel, the vacuum in the channel increases. This increased vacuum pressure can create a potentially hazardous situation. Once the occlusion is relieved—often by the particle being sucked through or dislodged—the sudden release of vacuum pressure results in a rapid inflow of fluid into the aspiration channel. This rapid inflow is known as a post-occlusion surge (POS). The POS can cause a sudden movement of intraocular structures, such as the posterior capsule or the iris, potentially leading to complications such as capsular rupture or other damage within the eye. Controlling or minimizing post-occlusion surge is beneficial for maintaining the safety and efficacy of the phacoemulsification procedure.

When using a valve module, such as an Anti-Vacuum Surge (AVS) valve (as shown for example in FIG. 2), to rapidly close the aspiration channel during phacoemulsification, a potential issue arises with the method of actuating the valve. The inventors have found that, if a typical electromagnetic pulse driven by a rectangular pulse of current is used to control the valve's plunger, it may lead to a recoil effect. The recoil may occur when the plunger reaches the end of its motion and then bounces back slightly. The recoil might delay full closing of the aspiration channel or might prevent the AVS valve from fully closing the aspiration channel, thereby failing to effectively mitigate the POS.

This recoil effect compromises the valve's ability to maintain a stable vacuum, leading to inadequate control of fluid dynamics within the aspiration channel. As a result, the expected rapid closure to prevent or minimize the POS may not be achieved, potentially causing complications during the procedure.

In order to address this issue, the present disclosure provides a phacoemulsification system that includes a controller programmed to provide an electric current pulse with a specific shape. The current pulse comprises a first section configured to accelerate the plunger and a second section designed to control the plunger's speed as it approaches the end of its travel. This approach prevents the plunger from hitting the end of its motion too forcefully, thereby avoiding the recoil effect and enabling reliable closure of the AVS valve. This improved control over the plunger's motion enhances the system's ability to maintain a stable vacuum and effectively mitigate POS during the procedure.

### BRIEF DESCRIPTION OF THE DRAWINGS

Some examples of the disclosure are herein described, by way of example only, with reference to the accompanying drawings. With specific reference now to the drawings in detail, it is stressed that the particulars shown are by way of example and for purposes of illustrative discussion of examples of the disclosure. In this regard, the description taken with the drawings makes apparent to those skilled in the art how examples of the disclosure may be practiced.

FIG. 1 is a partly pictorial, partly block diagram view of a phacoemulsification system constructed and operative in accordance with an example of the present disclosure;

FIG. 2 shows a cross-section of a fluid dynamics cartridge/module through an aspiration line in accordance with an example of the present disclosure;

FIG. 3 is a simplified graph showing a rectangular pulse of current and a location of a plunger in accordance with an example of the present disclosure;

FIG. 4 is a simplified graph showing a rectangular pulse of current and a location of a plunger in accordance with an example of the present disclosure;

FIG. 5 is a simplified graph showing a trapezoidal shaped pulse of current and a location of a plunger in accordance with an example of the present disclosure;

FIG. 6 is a simplified graph showing a smooth curve shaped pulse of current and a location of a plunger in accordance with an example of the present disclosure;

FIG. 7 is a simplified flow chart illustration of a method for closing a valve module and preventing a recoil effect in accordance with an example of the present disclosure;

FIG. 8 is a simplified flow chart illustration of a method of selecting a shape of a current pulse used for closing a valve module in a phacoemulsification probe in a phacoemulsification system and preventing a recoil effect in accordance with an example of the present disclosure; and

FIG. 9 is a simplified flow chart illustration of a method of selecting a shape of a current pulse used for closing a valve module in a phacoemulsification probe in a phacoemulsification system and reducing a recoil effect in accordance with an example of the present invention.

### DETAILED DESCRIPTION

The present disclosure relates to a phacoemulsification apparatus including a valve module such as an Anti-Vacuum Surge (AVS) module (also known as Chamber Stabilization System (CSS)), and, more particularly, but not exclusively, to an AVS valve mechanism enabling closing the valve as fast as possible while optionally preventing a recoil effect.

The term “plunger” may also be referred to throughout the present specification and claims as “valve closing element”.

### Introduction

Typical phacoemulsification systems have some type of way to minimize vacuum surge, also called post occlusion surge. For example, an AVS mechanism, valve module or device is configured to stop vacuum operation to prevent eye damage.

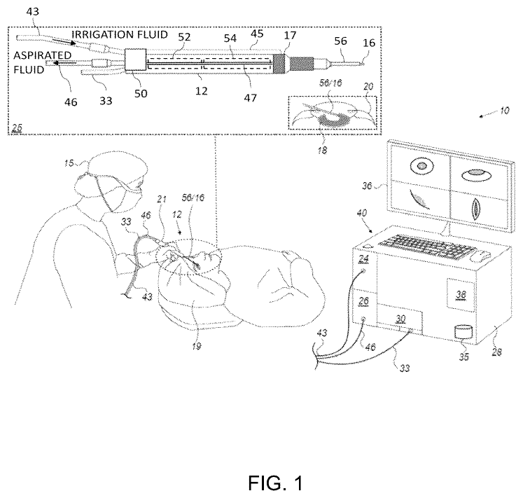

Reference is now made to FIG. 1, which is a partly pictorial, partly block diagram view of a phacoemulsification system constructed and operative in accordance with an example of the present disclosure.

FIG. 1 shows a phacoemulsification system 10 comprising a phacoemulsification probe 12 (e.g., handpiece). In some examples, the phacoemulsification probe 12 may be replaced by any suitable medical tool. As seen in the pictorial view of phacoemulsification system 10, and in inset 25, phacoemulsification probe 12 comprises a needle 16, a probe body 17, and a coaxial irrigation sleeve 56 that at least partially surrounds needle 16 and creates a fluid pathway between the external wall of the needle and the internal wall of the irrigation sleeve, where needle 16 is hollow to provide an aspiration channel. Moreover, irrigation sleeve 56 may have one or more side ports at, or near, the distal end to allow irrigation fluid to flow towards the distal end of the phacoemulsification probe 12 through the fluid pathway and out of the port(s).

Needle 16 is configured for insertion into a lens capsule 18 of an eye 20 of a patient 19 by a physician 15 to remove a cataract. While the needle 16 (and irrigation sleeve 56) are shown in inset 25 as a straight object, any suitable needle may be used with phacoemulsification probe 12, for example, a curved or bent tip needle commercially available from Johnson & Johnson Surgical Vision, Inc., Irvine, Calif., USA.

In the example of FIG. 1, during the phacoemulsification procedure, a pumping sub-system 24 comprised in a console 28 pumps irrigation fluid from an irrigation reservoir (not shown) to the irrigation sleeve 56 to irrigate the eye 20. The irrigation fluid is pumped via an irrigation tubing line 43 running from the console 28 to an irrigation channel 45 of probe 12, the distal end of the irrigation channel 45 including the fluid pathway in the irrigation sleeve 56. The irrigation tubing line 43 is typically flexible and may be prone to collapsing during an occlusion of the needle 16. In another example, the pumping sub-system 24 may be coupled or replaced with a gravity fed irrigation source such as a balanced salt solution bottle/bag.

Fluid and waste matter (e.g., emulsified parts of the cataract) are aspirated via an aspiration channel 47, which extends from the hollow of needle 16 through the phacoemulsification probe 12, and then via an aspiration tubing line 46 to a collection receptacle (not shown) in the console 28. The aspiration is affected by a pumping sub-system 26, also comprised in console 28.

System 10 may include a fluid dynamics cartridge 50 (which in an example, may be removable), which may include one or more valves to regulate the flow of fluid in the irrigation channel 45 and/or aspiration channel 47 as well as sensors. Part of the irrigation channel 45 and the aspiration channel 47 is disposed in the probe body 17 and part is disposed in the cartridge 50.

The fluid dynamics cartridge 50 shown in FIG. 1 is an example of a valve module or an AVS module. The AVS module may be located in the phacoemulsification probe 12 (e.g., handpiece), may be coupled with the proximal end of the handpiece, or may be coupled with the irrigation/aspiration lines near the proximal end of the handpiece. Examples of the present invention are also applicable to just a valve having the features described.

Phacoemulsification probe 12 includes other elements, such as one or more piezoelectric crystal(s) 52 coupled to a horn 54 to drive vibration of needle 16. The piezoelectric crystal(s) is configured to vibrate needle 16 in a resonant vibration mode. The vibration of needle 16 is used to break a cataract into small pieces during a phacoemulsification procedure. Console 28 comprises a piezoelectric drive module 30, coupled with the piezoelectric crystal 52, using electrical wiring running in a cable 33. Drive module 30 is controlled by a controller 38 and conveys processor-controlled driving signals via cable 33 to, for example, maintain needle 16 at maximal vibration amplitude. The drive module may be realized in hardware or software, for example, in a proportional-integral derivative (PID) control architecture. The controller 38 may also be configured to receive signals from sensors in the phacoemulsification probe 12 and/or fluid dynamics cartridge, e.g., AVS, and control one or more valves to regulate the flow of fluid in the irrigation channel 45 and/or the aspiration channel 47. In some examples, at least some of the functionality of the controller 38 may be implemented using a controller disposed in the phacoemulsification probe 12 (e.g., the cartridge 50).

Controller 38 may receive user-based commands via a user interface 40, which may include setting a vibration mode and/or frequency of the piezoelectric crystal(s) 52 and setting or adjusting an irrigation and/or aspiration rate of the pumping sub-systems 24/26. In some examples, user interface 40 and a display 36 may be combined as a single touch screen graphical user interface. In some examples, the physician 15 uses a foot pedal (not shown) as a means of control. Additionally, or alternatively, controller 38 may receive the user-based commands from controls located in a handle 21 of probe 12.

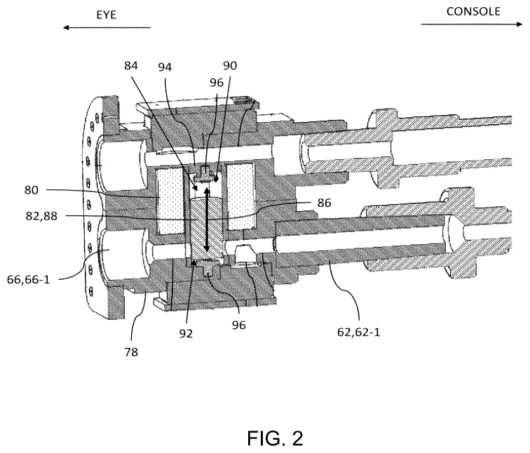

Reference is now made to FIG. 2, which shows a cross-section of a fluid dynamics cartridge including a valve for closing and opening an aspiration line in accordance with an example of the present invention.

FIG. 2 shows a valve body 78 includes ports 62, ports 66, a valve cavity 84 having a direction of elongation 86 and configured to provide fluid connectivity between respective ones of the ports 62, 66 (e.g., between an inlet port 66-1 and an outlet port 62-1). A solenoid coil 80 is disposed in the valve body 78 around valve cavity 84. A plunger 82 includes a permanent magnet 88. The permanent magnet 88 may comprise all of, or only part of, the plunger 82. For example, the plunger 82 may include the permanent magnet 88 coated or covered with a material of low friction. The plunger 82 is configured to move back-and-forth along the direction of elongation 86 between an (open) position 90 and a (closed) position 92 in the valve cavity 84 selectively controlling the fluid connectivity between respective ones of the ports 62, 66 (e.g., between the inlet port 66-1 and outlet port 62-1).

The valve body 78 may include a spacer 94. The valve body 78 may also include one or more dampers 96 to soften banging of the plunger 82 against the valve body 78. In FIG. 2, the upper damper 96 forms part of the spacer 94.

A controller may be configured to apply current to the solenoid coil 80 to cause an electromagnetic field to selectively move the plunger 82 between the open position 90 and the closed position 92. The controller may also be configured to apply current to the solenoid coil to selectively maintain the plunger in the open position 90 and/or the closed position 92. Alternatively, or additionally, a latch mechanism (not shown) may maintain the plunger 82 in position after movement in the open position 90 or closed position 92.

The terms “current” and “current pulse” are used throughout the present specification and claims to imply voltage and voltage pulse. Applying voltage causes current to flow through a device, and causing current to flow through a device implies that voltage is applied to the device, as is known in the art.

Remarks about Electromagnetic Pulse Shapes

One approach to closing a valve as quickly as possible is to apply maximum current to a solenoid affecting the closing of the valve until the valve is closed. Such an approach produces what can be called a rectangular pulse of current, starting when a command to close the valve is initiated, until the valve is closed.

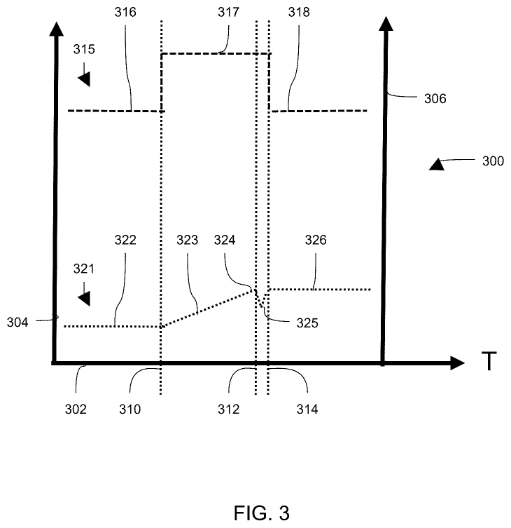

Reference is now made to FIG. 3, which is a simplified graph showing a rectangular pulse of current and a location of a plunger in accordance with an example of the present invention.

FIG. 3 is intended to show behavior of a plunger using an electromagnetic pulse driven with a rectangular pulse of current, where the plunger bounces back after reaching an end of its travel.

FIG. 3 is a graph 300 with an X-axis 302 showing qualitative time, and two Y-axes-a left Y-Axis 304 showing qualitative value of electric current or electric voltage applied to a solenoid for closing a plunger, and a right Y-axis 306 showing qualitative value of the location of a plunger.

A first (upper) dashed line 315 shows a first section 316 showing a qualitative value of electric current applied to a solenoid for closing a plunger, where the qualitative value may be zero, negative or a low value, and a second (lower) dotted line 321 showing a first section 322 showing a location of the plunger which corresponds to an open location.

The first dashed line 315 shows a second section 317 starting at a first time 310 when a value of current is applied to the solenoid, in order to move the plunger to close the valve, and the second dotted line 321 shows a second section 323 where the plunger is accelerated toward a closed position.

The first dashed line 315 shows the second section 317 reaching a second time 312 and a third time 314, where the same value of the current is still applied to the solenoid.

Over the same time span the second dotted line 321 shows a point 324 at the second time 312, where the plunger hits a closed position, followed by the plunger bouncing back to a non-closed position 325, and being accelerated again to a closed position 326 up to the third time 314.

Once the plunger closes the valve, for example at time 314, the value of current that is applied to the solenoid, as is shown by a third section 318 of the first dashed line 315, may be lowered, maintaining the closed position of the valve. The value of current that is applied to the solenoid to keep it closed may be zero, positive, or negative, as the case may be for a specific valve.

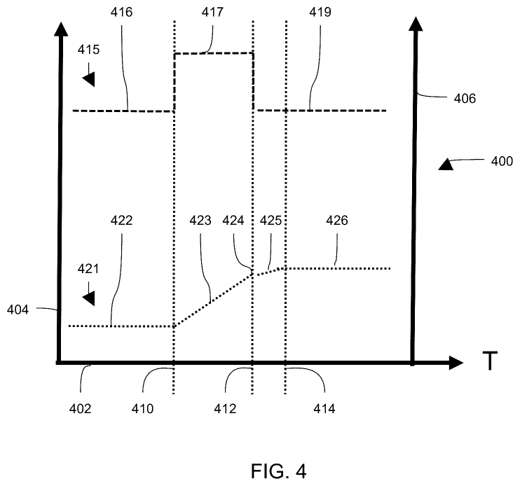

Reference is now made to FIG. 4, which is a simplified graph showing a rectangular pulse of current and a location of a plunger in accordance with an example of the present invention.

FIG. 4 is intended to show behavior of a plunger using an electromagnetic pulse driven with a rectangular pulse of current, where the rectangular pulse ends before the plunger reaches an end of its travel.

FIG. 4 is a graph 400 with an X-axis 402 showing qualitative time, and two Y-axes-a left Y-Axis 404 showing qualitative value of electric current or electric voltage applied to a solenoid for closing a plunger, and a right Y-axis 406 showing qualitative value of the location of a plunger.

A first dashed line 415 shows a first section 416 showing a qualitative value of electric current applied to a solenoid for closing a plunger, where the qualitative value may be zero, negative or a low value, and a second dotted line 421 showing a first section 422 showing a location of the plunger which corresponds to an open location.

The first dashed line 415 shows a second section 417 starting at a first time 410 when a value of current is applied to the solenoid, in order to move the plunger to close the valve, and the second dotted line 421 shows a second section 423 where the plunger is accelerated toward a closed position.

The first dashed line 415 shows the second section 417 reaching a second time 412, and at the second time 412 the value of current applied to the solenoid is lowered, as shown by a third section 419. As noted with reference to FIG. 3, the value of current that is applied to the solenoid to keep it closed may be zero, positive, or negative, as the case may be for a specific valve.

Over the same time span, when the second dotted line 421 reaches the second time 412, acceleration of the plunger is lowered, as shown by a third section 425 at a lower angle than the second section 423. The plunger does not yet reach the closed position, and its movement slows or decelerates, potentially by friction, until it reaches a closed level 426 at a third time 414.

Once the plunger closes the valve, for example at the third time 414, the value of current is applied to the solenoid may be lowered even more (not shown), maintaining the closed position of the valve.

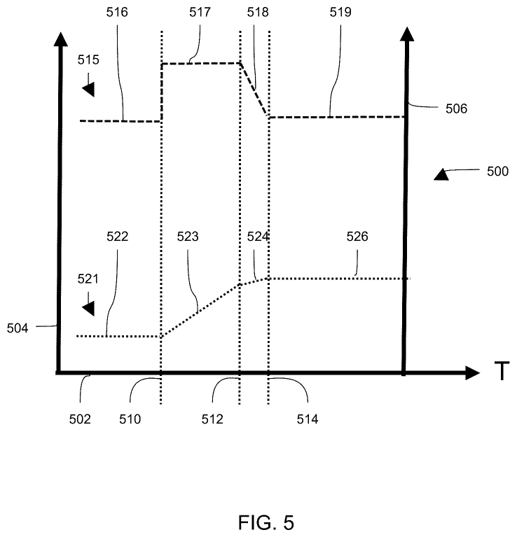

Reference is now made to FIG. 5, which is a simplified graph showing a trapezoidal shaped pulse of current and a location of a plunger in accordance with an example of the present invention.

FIG. 5 is intended to show behavior of a plunger using an electromagnetic pulse driven with a trapezoidal shaped pulse of current, where a rectangular pulse ends before the plunger reaches an end of its travel, and the current is reduced in a linear, non-abrupt fashion.

FIG. 5 is a graph 500 with an X-axis 502 showing qualitative time, and two Y-axes-a left Y-Axis 504 showing qualitative value of electric current or electric voltage applied to a solenoid for closing a plunger, and a right Y-axis 506 showing qualitative value of location of a plunger.

A first dashed line 515 shows a first section 516 showing a qualitative value of electric current applied to a solenoid for closing a plunger, where the qualitative value may be zero, negative or a low value, and a second dotted line 521 showing a first section 522 showing a location of the plunger which corresponds to an open location.

The first dashed line 515 shows a second section 517 starting at a first time 510 when a value of current is applied to the solenoid, in order to move the plunger to close the valve, and the second dotted line 521 shows a second section 523 where the plunger is accelerated toward a closed position.

The first dashed line 515 shows the second section 517 reaching a second time 512, and at the second time 512 the value of current applied to the solenoid is gradually reduced, in a linear, non-abrupt fashion, as shown by a third section 518, down to a level of a fourth section 519.

Over the same time span, when the second dotted line 521 reaches the second time 512, acceleration of the plunger is lowered, as shown by a third section 524 at a lower angle than the second section 523. The plunger does not yet reach the closed position, and its movement is less accelerated than during an initial time shown by the second section 523, and slows, until it reaches a closed level 526 at a third time 514.

Once the plunger closes the valve, for example at the third time 514, the value of current is applied to the solenoid may be lowered even more (not shown), maintaining the closed position of the valve. As noted with reference to FIGS. 3 and 4, the value of current that is applied to the solenoid to keep it closed may be zero, positive, or negative, as the case may be for a specific valve.

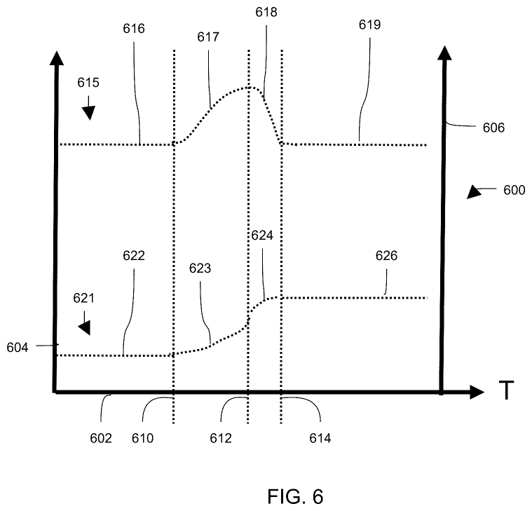

Reference is now made to FIG. 6, which is a simplified graph showing a smooth curve shaped pulse of current and a location of a plunger in accordance with an example of the present invention.

FIG. 6 is intended to show behavior of a plunger using an electromagnetic pulse driven with a smooth curve shaped pulse of current, where a current peak ends before the plunger reaches an end of its travel, and the current is also reduced in a smooth curve fashion.

FIG. 6 is a graph 600 with an X-axis 602 showing qualitative time, and two Y-axes-a left Y-Axis 604 showing qualitative value of electric current or electric voltage applied to a solenoid for closing a plunger, and a right Y-axis 606 showing qualitative value of location of a plunger.

A first dashed line 615 shows a first section 616 showing a qualitative value of electric current applied to a solenoid for closing a plunger, where the qualitative value may be zero, negative or a low value, and a second dotted line 621 showing a first section 622 showing a location of the plunger which corresponds to an open location.

The first dashed line 615 shows a second section 617 starting at a first time 610 when a value of current is applied to the solenoid, and increasing as time increases, in order to move the plunger to close the valve, and the second dotted line 621 shows a second section 623 where the plunger is accelerated toward a closed position.

The first dashed line 615 shows the second section 617 peaking at a second time 612, and at the second time 612 the value of current applied to the solenoid is reduced, in a smooth curve shaped, non-abrupt fashion, as shown by a third section 618.

Over the same time span, when the second dotted line 621 reaches the second time 612, acceleration of the plunger is reduced, as shown by a third section 624 at a lower angle than the second section 623. The plunger does not yet reach the closed position, and its movement is less accelerated than during an initial time shown by the second section 623, and slows, until it reaches a closed level 626 at a third time 614.

Once the plunger closes the valve, for example at the third time 614, the value of current is applied to the solenoid may be lowered even more (not shown), maintaining the closed position of the valve. As noted with reference to FIGS. 3, 4, and 5, the value of current that is applied to the solenoid to keep it closed may be zero, positive, or negative, as the case may be for a specific valve.

Remarks about Complete and Incomplete Closing of the Valve

It is noted that in order to minimize or eliminate effects of a post occlusion surge, it may not be necessary to completely close the valve. It may be sufficient to almost close the valve.

Referring to FIGS. 3, 4, 5, and 6 above, it may be enough to stop acting upon the plunger in a direction of closing the valve before the valve completely closes, resulting in incomplete closing of the valve, which may be sufficient to prevent lasting medical effects on a patient's eye of a post occlusion surge.

Remarks about Calibrating Current or Voltage Pulse Shape Combinations

In some examples, a shape of a current or voltage pulse to be used for closing a valve may optionally be modified in a laboratory setting to achieve desired results such as, by way of some non-limiting examples, closing a valve in a shortest time, or rapidly reducing suction pressure caused to an eye by an incident of a post occlusion surge, or determining how close to complete closing of a valve is sufficient to reduce suction pressure to a safe level following an incident of a post occlusion surge.

A non-limiting example of calibrating a specific valve may be calibrating a valve in a phacoemulsification probe 12 such as shown in FIG. 1.

A scenario of a vacuum surge may be artificially produced in a laboratory setting, where an eye side of a valve 66-1 in a fluid dynamics cartridge 50 (see FIG. 1) is connected to provide irrigation fluid, and a console side 62-1 of the valve in the fluid dynamics cartridge 50 is connected to an aspiration pump.

In preparation for simulating an unblocking of an aspiration channel, the eye side of the valve is blocked, simulating the aspiration channel being blocked.

At a time, for example TO, the eye side of the valve is unblocked, and a specific shape of a current or voltage pulse is used to close the valve.

Time is measured between a start of the current or voltage pulse T1 and a time T2 when closing of the valve is detected.

Various shapes of current or voltage pulses are optionally used to close the valve, and a shape providing the shortest duration for closing (T2-T1) may optionally be selected as a shape to be used for closing the specific valve used in the calibration, for example an aspiration valve of the phacoemulsification probe.

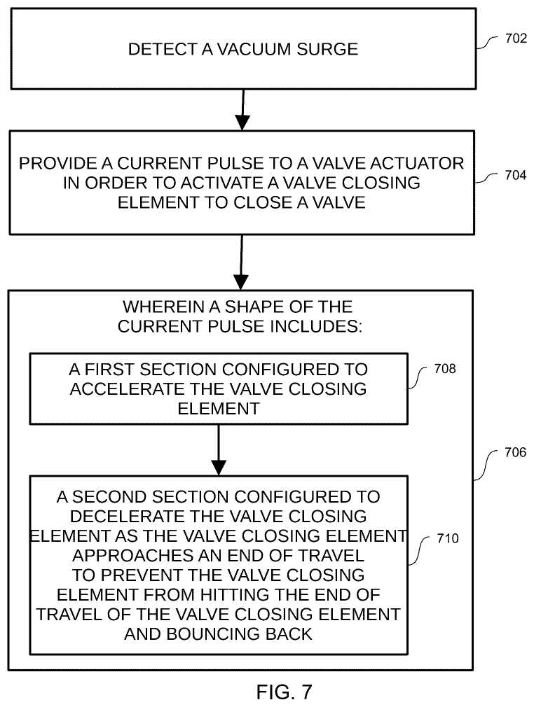

Reference is now made to FIG. 7, which is a simplified flow chart illustration of a method for closing a valve module in a phacoemulsification probe in accordance with an example of the present invention.

The method illustrated by FIG. 7 includes:
detecting a vacuum surge (702);
providing a current pulse to a valve actuator in order to activate a valve closing element to close a valve (704);
wherein a shape of the current pulse comprises (706):
a first section configured to accelerate the valve closing element (708); and
a second section configured to decelerate the valve closing element as the valve closing element approaches an end of travel to prevent the valve closing element from hitting the end of travel of the valve closing element and bouncing back (710), thereby preventing a recoil effect.

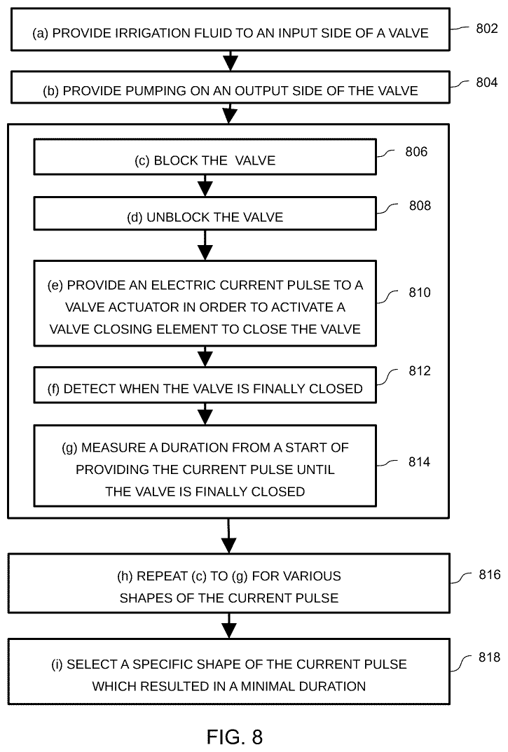

Reference is now made to FIG. 8, which is a simplified flow chart illustration of a method of selecting a shape of a current pulse used for closing a valve module in a phacoemulsification probe in a phacoemulsification system and preventing a recoil effect in accordance with an example of the present invention.

The method illustrated by FIG. 8 includes:
(a) providing irrigation fluid to an input side of a valve (802);
(b) providing pumping on an output side of the valve (804);
(c) blocking the valve (806);
(d) unblocking the valve (808);
(e) providing an electric current pulse to a valve actuator in order to activate a valve closing element to close the valve (810);
(f) detecting when the valve is finally closed (812);
(g) measuring a duration from a start of providing the current pulse until the valve is finally closed (814);
(h) repeating (c) to (g) for various shapes of the current pulse (816); and
(i) selecting a specific shape of the current pulse which resulted in a minimal duration (818).

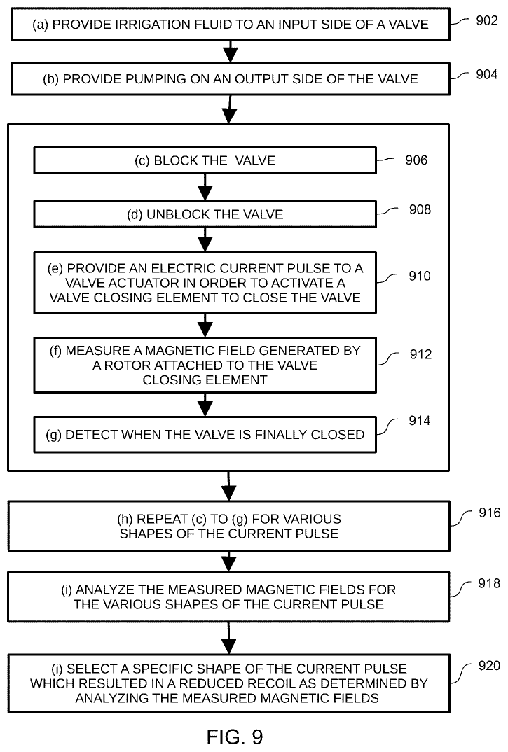

Reference is now made to FIG. 9, which is a simplified flow chart illustration of a method of selecting a shape of a current pulse used for closing a valve module in a phacoemulsification probe in a phacoemulsification system and reducing a recoil effect in accordance with an example of the present invention.

The method illustrated by FIG. 9 includes:
(a) providing irrigation fluid to an input side of a valve (902);
(b) providing pumping on an output side of the valve (904);
(c) blocking the valve (906);
(d) unblocking the valve (908);
(e) providing an electric current pulse to a valve actuator in order to activate a valve closing element to close the valve (910);
(f) measuring a magnetic field generated by a rotor attached to the valve closing element (912)
(g) detecting when the valve is finally closed (914);
(h) repeating (c) to (g) for various shapes of the current pulse (916);
(i) analyzing the measured magnetic fields for the various shapes of the current pulse (918); and
(j) selecting a specific shape of the current pulse which results in a reduced recoil as determined by analyzing the measured magnetic fields (920).

In some examples, a shape of a current or voltage pulse to be used for closing a valve may optionally be modified in a real use setting.

A controller (such as shown in FIG. 1) may optionally be configured to detect a valve closing element bouncing back, for example by analyzing pressure over time, or flow rate over time, or by measuring and analyzing the magnetic field generated by a rotor attached to the valve closing element.

When the controller detects the valve closing element bouncing back, the controller may optionally change the shape of the current pulse, to prevent the valve closing element from bouncing back. Some non-limiting examples of such changes include:
ending an acceleration section of the current pulse earlier, e.g. making the second sections 417, 517 shown in FIGS. 4 and 5 shorter;
making a deceleration section of the current pulse to decelerate the valve closing element more, e.g. making the third section 419 shown in FIG. 4 lower, or. making the fourth section 519 shown in FIG. 5 lower, or making the slope of the third section 518 shown in FIG. 5 to be more abrupt; and
making a deceleration section of the current pulse start earlier, e.g. shifting the second time 612 shown in FIG. 6 to begin an earlier reduction of acceleration and start of deceleration.
   Remarks about Detecting Vacuum Surge and Detecting when an AVS Valve is Closed

In some examples, a sensor for detecting a vacuum surge is constructed within a phacoemulsification probe.

In some examples, a sensor for detecting a vacuum surge is constructed externally to the phacoemulsification probe, by way of a non-limiting example on an aspiration channel, in some cases close to the phacoemulsification probe, in some cases close to an aspiration pump.

In some examples, the sensor and the AVS valve are optionally both constructed within the phacoemulsification probe.

Sensors as mentioned above may be any sensor known in the art which can detect one or more of: vacuum; a slowing of aspiration flow; or effect of the vacuum surge on an eye—for example deformation of the cornea.

Pressure and flow sensors include, by way of some non-limiting examples: a vacuum sensor; a pressure sensor; a flow sensor.

The pressure and flow sensors' sensor measurements (e.g., pressure, vacuum, and/or flow) may be taken close to a distal end of a phacoemulsification probe where an irrigation outlet and an aspiration inlet are located, so as to provide an accurate indication of measurements occurring within an eye and optionally provide a short response time to a control loop comprised in a processor.

It is noted that other types of sensors such as temperature sensors, optical sensors, capacitance sensors and/or other sensors may also be incorporated as part of the circuitry of the system.

One or more of the above-mentioned sensors may also be used to detect when an AVS valve is closed—leading to no flow in the aspiration line.

Measurement of deformation of the cornea may optionally be made by shining laser light upon the cornea and tracking a shape of a reflection of the laser beam.

A beginning of deformation of the cornea may optionally indicate a vacuum surge.

Following activation of a current pulse to close the AVS valve, an end to deformation of the cornea may optionally indicate a closing of the valve, or at least a valve that is closed enough to mitigate the undesired effect of a vacuum surge on the cornea.

### EXAMPLES

### Example 1

A phacoemulsification system (10) comprising a phacoemulsification probe (12) including a hollow needle (16) configured to provide an aspiration channel (47), a valve module (78) including a valve closing element (82) configured to close the aspiration channel upon activation, and a controller (38) for providing an electric current pulse to activate the valve closing element to close the valve module, wherein the controller is programmed to detect a vacuum surge, and provide an electric current pulse to a valve actuator in order to activate the valve closing element to close the valve module, and wherein the shape of the current pulse includes a first section (417, 517, 617) configured to accelerate the valve closing element, and a second section (419, 518, 618) configured to decelerate the valve closing element as the valve closing element approaches an end of travel to prevent the valve closing element from hitting the end of travel of the valve closing element and bouncing back, thereby preventing a recoil effect.

### Example 2

The system according to example 1, wherein the valve module is included in the phacoemulsification probe.

### Example 3

The system according to any one of examples 1-2, wherein the system includes a sensor for detecting deformation of the cornea.

### Example 4

The system according to any one of examples 1-3, wherein the phacoemulsification probe includes a sensor for detecting the vacuum surge.

### Example 5

The system according to any one of examples 1-4 wherein the first section (417) has a constant current value ending before the valve closing element reaches the end of travel of the valve closing element, and the second section (419) has a constant current value lower than the first section.

### Example 6

The system according to any one of examples 1-4, wherein the first section (517) has a constant current value ending before the valve closing element reaches the end of travel of the valve closing element, and the second section (518) includes a linear reduction of current.

### Example 7

The system according to any one of examples 1-4, wherein the first section (617) includes a smooth increase of the current, and the second section (618) includes a smooth decrease of current.

### Example 8

The system according to any one of examples 1-7, wherein the shape of a current pulse used for closing the AVS valve includes a shape which has been determined in a calibration laboratory to provide minimal closing time and also prevent the valve closing element from bouncing back.

### Example 9

A method for closing a valve and preventing a recoil effect, the method comprising detecting a vacuum surge, providing a current pulse to a valve actuator in order to activate a valve closing element (82) to close a valve (78), wherein a shape of the current pulse used for closing the valve includes a first section (417, 517, 617) configured to accelerate the valve closing element, and a second section (419, 518, 618) configured to decelerate the valve closing element as the valve closing element approaches an end of travel to prevent the valve closing element from hitting the end of travel of the valve closing element and bouncing back, thereby preventing a recoil effect.

### Example 10

The method according to example 9 wherein the valve is included in a phacoemulsification system.

### Example 11

The method according to any one of examples 9-10 wherein the detecting the vacuum surge includes detecting the vacuum surge in a phacoemulsification probe.

### Example 12

The method according to any one of examples 9-11 wherein the first section (417) has a constant current value ending before the valve closing element reaches the end of travel of the valve closing element, and the second section (419) has a constant current value lower than the first section.

### Example 13

The method according to any one of examples 9-11, wherein the first section (517) has a constant current value ending before the valve closing element reaches the end of travel of the valve closing element, and the second section (518) includes a linear reduction of current.

### Example 14

The method according to any one of examples 9-11, wherein the first section (617) includes a smooth increase of the current, and the second section (618) includes a smooth decrease of current.

### Example 15

The method according to any one of examples 9-14, wherein the shape of the current pulse used for closing the valve includes a shape which has been determined in a calibration laboratory to provide minimal closing time.

### Example 16

The method according to any one of examples 9-14, wherein the shape of the current pulse used for closing the valve includes a shape which has been determined in a calibration laboratory to prevent the valve closing element from bouncing back.

### Example 17

A computer-readable storage medium including instructions which, when executed by a computer, cause the computer to carry out the method of example 9.

### Example 18

A method of selecting a shape of a current pulse used for closing a valve in a phacoemulsification system and preventing a recoil effect, the method comprising:
(a) providing irrigation fluid to an input side of a valve (802),
(b) providing pumping on an output side of the valve (804),
(c) blocking the valve (806),
(d) unblocking the valve (808),
(e) providing an electric current pulse to a valve actuator in order to activate a valve closing element to close the valve (810),
(f) detecting when the valve is finally closed (812),
(g) measuring a duration from a start of providing the current pulse until the valve is finally closed (814),
(h) repeating (c) to (g) for various shapes of the current pulse (816), and
(i) selecting a specific shape of the current pulse which resulted in a minimal duration (818).

### Example 19

The method according to example 18, wherein the detecting when the valve is finally closed is performed by a sensor included in the phacoemulsification system.

### Example 20

The method according to any one of examples 18-19, wherein the detecting when the valve is finally closed is performed by a sensor included in the phacoemulsification probe.

### Example 21

A computer-readable storage medium including instructions which, when executed by a computer, cause the computer to carry out the method of example 18.

### Example 22

A method of selecting a shape of a current pulse used for closing a valve in a phacoemulsification system and preventing a recoil effect, the method comprising:
(a) providing irrigation fluid to an input side of a valve (902),
(b) providing pumping on an output side of the valve (904),
(c) blocking the valve (906),
(d) unblocking the valve (908),
(e) providing an electric current pulse to a valve actuator in order to activate a valve closing element to close the valve (910),
(f) measuring a magnetic field generated by a rotor attached to the valve closing element (912),
(g) detecting when the valve is finally closed (914),
(h) repeating (c) to (g) for various shapes of the current pulse (916),
(i) analyzing the measured magnetic fields for the various shapes of the current pulse (918), and
(j) selecting a specific shape of the current pulse which results in a reduced recoil as determined by analyzing the measured magnetic fields (920).

As such, those skilled in the art to which the present invention pertains, can appreciate that while the present invention has been described in terms of preferred examples, the concept upon which this disclosure is based may readily be utilized as a basis for the designing of other structures, systems and processes for carrying out the several purposes of the present invention.

Also, it is to be understood that the phraseology and terminology employed herein are for the purpose of description and should not be regarded as limiting.

It should be noted that the words “comprising”, “including” and “having” as used throughout the appended claims are to be interpreted to mean “including but not limited to”. The indefinite articles “a” and “an,” as used herein in the specification and in the claims, unless clearly indicated to the contrary, should be understood to mean “at least one.” The phrase “and/or” as used herein in the specification and in the claims, should be understood to mean “either or both” of the elements so conjoined, i.e., elements that are conjunctively present in some cases, and disjunctively present in other cases.

It is important, therefore, that the scope of the invention is not construed as being limited by the illustrative examples set forth herein. Other variations are possible within the scope of the present invention as defined in the appended claims. Other combinations and sub-combinations of features, functions, elements and/or properties may be claimed through amendment of the present claims or presentation of new claims in this or a related application. Such amended or new claims, whether they are directed to different combinations or directed to the same combinations, whether different, broader, narrower or equal in scope to the original claims, are also regarded as included within the subject matter of the present description.

It is expected that during the life of a patent maturing from this application many relevant valves will be developed and the scope of the term valve is intended to include all such new technologies a priori.

The terms “comprising”, “including”, “having” and their conjugates mean “including but not limited to”.

As used herein, the singular form “a”, “an” and “the” include plural references unless the context clearly dictates otherwise.

The term “example” is used herein to mean “serving as an example, instance or illustration”. Any example described is not necessarily to be construed as preferred or advantageous over other examples and/or to exclude the incorporation of features from other examples.

The word “optionally” is used herein to mean “is provided in some examples and not provided in other examples”. Any particular example of the disclosure may include a plurality of “optional” features unless such features conflict.

Throughout this application, various examples of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range, including minimum and maximum values of the range and fractional values in the range.

Unless otherwise indicated, numbers used herein, and any number ranges based thereon are approximations within the accuracy of reasonable measurement and rounding errors as understood by persons skilled in the art.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. Certain features described in the context of various examples are not to be considered essential features of those examples, unless the example is inoperative without those elements.

To the extent that section headings are used, they should not be construed as necessarily limiting.

1. A phacoemulsification system comprising:
a phacoemulsification probe comprising a hollow needle configured to provide an aspiration channel;
a valve module comprising a valve closing element configured to close the aspiration channel upon activation; and
a controller for providing an electric current pulse to activate the valve closing element to close the valve module,
wherein the controller is programmed to:
detect a vacuum surge; and
provide an electric current pulse to a valve actuator in order to activate the valve closing element to close the valve module, and
wherein the shape of the current pulse comprises:
a first section configured to accelerate the valve closing element; and
a second section configured to decelerate the valve closing element as the valve closing element approaches an end of travel to prevent the valve closing element from hitting the end of travel of the valve closing element and bouncing back, thereby preventing a recoil effect.

2. The system according to claim 1, wherein the valve module is comprised in the phacoemulsification probe.

3. The system according to claim 1, wherein the system comprises a sensor for detecting deformation of the cornea.

4. The system according to claim 1, wherein the phacoemulsification probe comprises a sensor for detecting the vacuum surge.

5. The system according to claim 1, wherein the first section has a constant current value ending before the valve closing element reaches the end of travel of the valve closing element; and the second section has a constant current value lower than the first section.

6. The system according to claim 1, wherein the first section has a constant current value ending before the valve closing element reaches the end of travel of the valve closing element; and the second section comprises a linear reduction of current.

7. The system according to claim 1, wherein the first section comprises a smooth increase of the current; and the second section comprises a smooth decrease of current.

8. The system according to claim 1, wherein the shape of a current pulse used for closing the AVS valve comprises a shape which has been determined in a calibration laboratory to provide minimal closing time and also prevent the valve closing element from bouncing back.

9. A method for closing a valve and preventing a recoil effect, the method comprising:
detecting a vacuum surge;
providing a current pulse to a valve actuator in order to activate a valve closing element to close a valve;
wherein a shape of the current pulse used for closing the valve comprises:
a first section configured to accelerate the valve closing element; and
a second section configured to decelerate the valve closing element as the valve closing element approaches an end of travel to prevent the valve closing element from hitting the end of travel of the valve closing element and bouncing back, thereby preventing a recoil effect.

10. The method according to claim 9, wherein the valve is comprised in a phacoemulsification system.

11. The method according to claim 9, wherein the detecting the vacuum surge comprises detecting the vacuum surge in a phacoemulsification probe.

12. The method according to claim 9, wherein the first section has a constant current value ending before the valve closing element reaches the end of travel of the valve closing element; and the second section has a constant current value lower than the first section.

13. The method according to claim 9, wherein the first section has a constant current value ending before the valve closing element reaches the end of travel of the valve closing element; and the second section comprises a linear reduction of current.

14. The method according to claim 9, wherein the first section comprises a smooth increase of the current; and the second section comprises a smooth decrease of current.

15. The method according to claim 9, wherein the shape of the current pulse used for closing the valve comprises a shape which has been determined in a calibration laboratory to provide minimal closing time.

16. The method according to claim 9, wherein the shape of the current pulse used for closing the valve comprises a shape which has been determined in a calibration laboratory to prevent the valve closing element from bouncing back.

17. A method of selecting a shape of a current pulse used for closing a valve in a phacoemulsification system and preventing a recoil effect, the method comprising:
(a) providing irrigation fluid to an input side of a valve;
(b) providing pumping on an output side of the valve;
(c) closing the valve;
(d) opening the valve;
(e) providing an electric current pulse to a valve actuator in order to activate a valve closing element to close the valve;
(f) detecting when the valve is finally closed;
(g) measuring a duration from a start of providing the current pulse until the valve is finally closed;
(h) repeating (c) to (g) for various shapes of the current pulse; and
(i) selecting a specific shape of the current pulse which resulted in a minimal duration.

18. The method according to claim 17, wherein the detecting when the valve is finally closed is performed by a sensor included in the phacoemulsification system.

19. A method of selecting a shape of a current pulse used for closing a valve in a phacoemulsification system and preventing a recoil effect, comprising:
(a) providing irrigation fluid to an input side of a valve;
(b) providing pumping on an output side of the valve;
(c) blocking the valve;
(d) unblocking the valve;
(e) providing an electric current pulse to a valve actuator in order to activate a valve closing element to close the valve;
(f) measuring a magnetic field generated by a rotor attached to the valve closing element
(g) detecting when the valve is finally closed;
(h) repeating (c) to (g) for various shapes of the current pulse;
(i) analyzing the measured magnetic fields for the various shapes of the current pulse; and
(j) selecting a specific shape of the current pulse which results in a reduced recoil as determined by analyzing the measured magnetic fields.
